(19) United States
(12) Patent Application Publication
(10) Pub. No.: US 2021/0079178 A1
(43) Pub. Date: Mar. 18, 2021

(54) POLYMERIC DEVICES AND METHODS OF MAKING

(71) Applicant: MicroOptx Inc., Maple Grove, MN (US)

(72) Inventors: Tingrui Pan, Woodland, CA (US); Edward Aaron Cohen, Columbia Heights, MN (US); Ben Bazor, Davis, CA (US); Gaomai Yang, Davis, CA (US)

(21) Appl. No.: 17/105,111

(22) Filed: Nov. 25, 2020

Related U.S. Application Data
(63) Continuation of application No. 16/323,481, filed on Feb. 5, 2019.
(60) Provisional application No. 62/371,706, filed on Aug. 5, 2016.

(51) Int. Cl. C08J 5/12; C08J 7/18; C08G 81/00; B81C 3/00; C08J 7/16; C08F 290/14; C08J 7/12; C09J 5/00; B81C 1/00; C08G 81/02

(52) U.S. Cl. CPC C08J 5/128; C08J 2433/16; C08G 81/00; B81C 3/001; C08J 7/16; C08F 290/141; C08J 5/12; C08J 7/123; C09J 5/00; B81C 1/00071; C08G 81/027; C08J 2367/02; C09J 2400/226; B81C 2201/034; B81C 2203/036; C08J 2433/14; C08J 7/18

(57) ABSTRACT

Some polymeric devices, as described herein, can be made of a first layer and a second layer bonded together with one or more microfluidic channels defined internal to the device. The first layer and the second layer may each include a substrate and a polymer bonded to the substrate. The two layers may be bonded through a polymer network that interpenetrates the polymers in the first and second layers. This disclosure also describes methods of bonding together polymeric articles. The methods include diffusing polymerizable monomers and radical forming initiators into the surfaces of one or both of the polymers, putting the surfaces into contact, and initiating polymerization to create a polymer network that interpenetrates the polymers.

### REFERENCE TO RELATED APPLICATIONS

This application is a continuation of U.S. application Ser. No. 16/323,481 filed on Feb. 5, 2019, which is a National Stage Application under 35 U.S.C. § 371 and claims the benefit of International Application No. PCT/US2017/045259, filed Aug. 3, 2017, which claims the benefit of U.S. Provisional Application Ser. No. 62/371,706, filed Aug. 5, 2016. The disclosures of the prior applications are considered part of (and are incorporated by reference in) the disclosure of this application.

### STATEMENT AS TO FEDERALLY SPONSORED RESEARCH

None.

### BACKGROUND

L. H. Sperling, “Interpenetrating Polymer Networks”, (in Klempner et al., Advances in Chemistry; American Chemical Society: Washington, D C, 1994)), characterizes interpenetrating polymer networks as combinations of two or more polymers, synthesized in juxtaposition.

US 2003/0218130 (Boschetti et al.) refers to a substrate having a polymerized, polysaccharide-based hydrogel attached to the surface.

US 2004/0162545 (Brown et al.) refers to aqueous humor flow control for managing intraocular pressure in an eye.

Kim et al., (“In Situ Photopolymerization of a Polymerizable Poly(ethylene glycol)-Covered Phospholipid Monolayer on a Methacryloyl-Terminated Substrate”, 402-751, Korea Langmuir, 2004, 20 (13), pp 5396-5402 DOI: 10.1021/Ia049959g Publication Date (Web): May 21, 2004 2004 American Chemical Society refers to a method of creating a photopolymerized PEG/phosopholipid monolayer on a methacryloyl-terminated substrate.

US 2009/0178934 (Jarvius et al.) refers to a microfluidic structure comprising a thermoplastic portion defining a microfluidic recess, a bonding layer on the thermoplastic portion and a siloxane elastomer portion covalently bonded to the bonding layer to seal the microfluidic recess.

US 2010/0207301 (Suh et al.) refers to a method of forming microchannels. The document states that UV curable polymer patterns are formed on a first substrate, then the UV curable polymer patterns and a second substrate are sealed together by electrostatic attraction. Then, a channel is formed by irradiating UV light.

US 2013/0184631 (Pinchuk) refers to a surgical kit including at least one instrument and at least one device. The document states that the instrument has a needle body used for a surgical passage through ocular tissue. The document further state that the device includes a flexible tube that defines a duct for diverting aqueous humor with an outer surface having a maximal cross-sectional dimension that is less than the maximal cross-sectional dimension of the body.

International Publication WO 2014/207619 (Delamarche et al.) refers to methods of fabrication of a microfluidic chip package or assembly with separable chips.

U.S. Pat. No. 9,044,301 (Pinchuk et al.) refers to a system including an elongated needle body and aqueous humor drainage device.

### SUMMARY

In one aspect provided herein is a method of grafting together two polymeric articles comprising: (a) providing first and second polymeric articles, each polymeric article having a polymer layer having a surface; (b) contacting the surfaces with components selected from: (i) a solvent; (ii) a radical forming initiator; and (iii) polymerizable monomers; such that each component is contacted with the surface of at least one of the polymer layers; (c) contacting the surfaces of the polymer layers with each other; and (d) initiating polymerization to produce a grafting polymer of the monomers, wherein the grafting polymer interpenetrates both polymer layers, thereby grafting the first polymeric article to the second polymeric article.

In one embodiment contacting the surfaces with components comprises contacting each surface with a solution comprising the solvent and either or both of the radical forming initiator and the polymerizable monomers and allowing the radical forming initiator and the polymerizable monomers to penetrate the polymer layer. In another embodiment the method further comprises removing the solvent by evaporation (e.g., vacuum or heating). In another embodiment contacting the surfaces comprises applying a solvent to at least one of the surfaces, wherein the applied solvent draws the polymerizable monomers and the radical forming initiator to the surfaces of the polymer layers. In another embodiment the polymer layer comprise a hydrogel. In another embodiment the polymer layers have anti-biofouling properties. In another embodiment the polymer layer comprises poly-PEG acrylate, polyacrylamide, poly(glycerol), poly(2-oxazoline), poly(hydroxyfunctional acrylate), poly(vinylpyrrolidone), peptides, or peptoids. In another embodiment the polymer layers comprise the same or different polymers. In another embodiment the surface of at least one of the polymer layers comprises one or more microfeatures. In another embodiment at least one microfeature is a microchannel. In another embodiment the polymerizable monomers comprise a polymerizable polyethylene glycol (“PEG”) (e.g., an acrylate-PEG-acrylate such as triethylene glycol dimethacrylate). In another embodiment polymerizable monomers comprise a zwitterion capable of free radical polymerization. In another embodiment the polymerizable monomers comprise N-(2-methacryloyloxy)ethyl-N, N-dimethylammonio propanesulfonate (“SPE”), N-(3-methacryloylimino)propyl-N, N-dimethylammonio propanesulfonate (“SPP”), 2-(methacryloyloxy)ethylphosphatidylcholine (“M PC”), or 3-(2′-vinyl-pyridinio)propanesulfonate (“SPV”)). In another embodiment the polymerizable monomers comprise Poly(2-oxazoline)s (e.g. Poly(2-ethyl-2-oxazoline)); Poly(hydroxyfunctional acrylates) (e.g. Poly(2-hydroxyethyl methacrylate); Poly(glycerol)); poly(vinylpyrrolidone); peptides or peptoids (e.g. Poly(amino acid) or Poly(peptoid)). In another embodiment the polymerizable monomers comprise a plurality of different polymerizable monomers, wherein polymerizable monomers polymerize to form a copolymer. In another embodiment the different polymerizable monomers comprise heterodisperse polyethylene glycol monomers. In another embodiment the polymerizable monomers comprise acrylamides, acrylates, allyls, vinylics or styrenics. In another embodiment the radical forming initiator is a photo initiator or a thermal initiator. In another embodiment the radical forming initiator is a photo initiator selected from 2-hydroxy-2-methylpropiophenone, benzoin ethers, benzyl ketals, a-dialkoxy-alkyl-phenones, a-hydroxy-alkyl-phenones, a-amino alkyl-phenones, acyl-phosphine oxides, benzophenones, benzoamines, thioxanthones, thioamines and titantocenes. In another embodiment the radical forming initiator is a thermal initiator selected from tert-Amyl peroxybenzoate, 4-4-Azobis(4-cyanovaleric acid), 1-1 ‘Azobis(cyclohexanecarbonitrile), 2-2’-Azobisisobutyronitrile, Benzoyl peroxide, 2,2-Bis(tertbutylperoxy) butane, 1,1-Bis(tert-butylperoxy)cyclohexane, 2,5-Bis(tert-butylperoxy)-2,5-di methyl hexane, 2,5-Bis(tert-Butylperoxy)-2,5-di methyl-3-hexyne, Bis(1-(tert-butylperoxy)-1-methylethyl)benzene, 1,1-Bis(tert-butylperoxy)-3,3,5-trimethylcyclohexane, tert-Butyl hydroperoxide, tert-Butyl peracetate, tert-Butyl peroxide, tert-Butyl peroxybenzoate, tertButylperoxy isopropyl carbonate, Cumene hydroperoxide, Cylcohexanone peroxide, Dicumyl peroxide, Lauroyl peroxide, 2,4-Pentanedione peroxide, Peracetic acid and Potassium persulfate. In another embodiment the polymeric articles comprise a solid substrate attached to the polymer.

In another aspect provided herein is a method of making a polymeric device comprising: (a) providing a first article and a second article, each article comprising a polymer layer bonded to a solid substrate; and (b) grafting the polymer layers together by the method described above. In one embodiment the first article and/or second article comprise a polymer layer covalently attached to the substrate. In another embodiment the first article and/or second article comprise a second polymer layer grafted to the existing polymer layer. In another embodiment the first article and second article are each prepared by a method comprising: (i) providing a solid substrate comprising a surface comprising polymerizable moieties; (ii) contacting the surface of the substrate with a composition comprising first polymerizable monomers and a radical forming initiator; (iii) initiating polymerization to produce a polymer of the monomers, wherein the polymer is covalently bound through the polymerizable moieties to the substrate. In another embodiment the polymeric device comprises at least one microfeature, e.g., a microchannel, disposed in the grafted polymer layers. In another embodiment the solid substrate comprises a material selected from polyethylene terephthalate (“PET”), Poly(methyl methacrylate) (“PMMA”), Polyurethane (“PU”), Polycarbonate (“PC”), Polyvinyl chloride (“PVC”), Polyvinyl alcohol (“PVA”), Polystyrene (“PS”), Polyethylene (“PE”), Polysulfone, Polyethersulfone (“PES”), Sulfonated Polyethersulfone (“SPES”), Polybutylene terephthalate (“PBT”), Polyhydroxyalkanoates (“PHAs”), Silicone, Parylene, Polyproylene (“PP”), Fluoropolymers, Polyhydroxylethylmethacrylate (“pHEMA”), Polyetherketoneketone (“PEKK”), Polyether ether ketone (“PEEK”), Polyaryletherketone (“PAEK”), Poly(lactic-co-glycolic acid) (“PLGA”), Polytetrafluoroethylene (“PTFE”), or Polyvinylidene fluoride (“PVDF”) and Polyacrylic acid (“PAA”). In another embodiment the polymerizable moiety comprises C═C groups (“sp2-hydridized carbon atoms”). In another embodiment the C═C group is a terminal acrylate group (e.g., an acryloxypropyl silane). In another embodiment providing the solid substrate comprises functionalizing a polymeric substrate with hydroxyl groups and coupling (3-acryloxypropyl) trichlorosilane through the hydroxyl groups. In another embodiment functionalizing comprises exposing a surface to a physical or chemical treatment for surface hydroxyl activation (e.g., exposure to plasma (e.g., 02 plasma, air plasma, nitrogen plasma, H2 plasma, H2+H2O plasma), ozone, peroxide or piranha solution). In another embodiment providing at least one article comprises providing a mold optionally having one or more microfeatures, filling the mold with the composition, overlaying the composition with the solid substrate, optionally aligning the mold and the solid substrate and initiating polymerization. In another embodiment the mold comprises polyvinyl alcohol (“PVA”) coated silicon, polydimethyl siloxane (“PDMS”), SU-8 epoxy, parylene, PTFE, dry film photoresist, or glass. In another embodiment the mold comprises a plurality of microfeature patterns, each pattern for one of a plurality of microdevices. In another embodiment the method further comprises cleaving the microfeatured chip (e.g., by mechanical or laser cutting) to produce a plurality of microdevices. In another embodiment at least one microfeature is a microchannel, wherein the microchannel has an inlet and outlet, each opening through the polymer. In another embodiment the microchannel has a length between about 0.5 mm and about 500 mm (e.g., between about 2 mm and about 5 mm). In another embodiment the microdevice has an elongated shape. In another embodiment, for at least one article, the composition is further contacted with a mold, wherein the mold imparts a microchannel to the polymer, whereby the ultimate device includes an enclosed microchannel.

In another aspect provided herein is a method of making a polymeric device comprising: (a) providing a first article and a second article, each article comprising a polymer layer bonded to a substrate, wherein the polymer layers are partially cured; (b) contacting the polymer of the first article with the polymer of the second article; and (c) initiating polymerization to covalently bond the articles through polymerization of monomers in the first and second article into common chains in a network polymer, wherein the first article is bonded to the second article. In one embodiment the first article and second article are provided by a method comprising: (i) providing a solid substrate comprising a surface comprising polymerizable moieties; (ii) contacting the surface of the substrate with a composition comprising polymerizable monomers and a radical forming initiator; and (iii) initiating polymerization to produce a polymer of the monomers for a time sufficient to initiate polymerization and to produce a partially cured polymer of the monomers, wherein the polymer is covalently bound through the polymerizable moieties to the substrate. In another embodiment at least one of the articles comprises one or more microfeatures in a surface of the polymer. In another embodiment the first article and/or second article comprise a polymer layer covalently attached to the substrate. In another embodiment the first article and/or second article comprise a polymer layer grafted to the substrate.

In another aspect provided herein is a method of making a polymeric device comprising: (a) providing a first article comprising a polymer layer bonded to a solid substrate, wherein the polymer layer comprises polymerizable monomers and, optionally, a radical forming initiator, diffused into a surface of the polymer layer; (b) providing a second article comprising a polymer layer bonded to a solid substrate, wherein the polymer layer is partially cured; and (c) contacting the polymer of the first article with the polymer of the second article and initiating polymerization to covalently bond the articles through polymerization of polymerizable monomers in the first and second article into common chains in a network polymer, wherein the first article is bonded to the second article. In one embodiment at least one of the first and second article comprises one or more microfeatures in a surface of the polymer. In another embodiment providing the first article and second article comprises: (a) providing a first article, wherein the first article is prepared by a method comprising: (i) providing a solid substrate comprising a surface comprising polymerizable moieties; (ii) contacting the surface of the substrate with a composition comprising polymerizable monomers and a radical forming initiator; (iii) initiating polymerization to produce a polymer of the monomers for a time sufficient to initiate polymerization and to produce a partially cured polymer of the monomers, wherein the polymer is covalently bound through the polymerizable moieties to the substrate; and (b) providing a second article, wherein the second article is prepared by a method comprising: (i) providing a solid substrate comprising a surface comprising polymerizable moieties; (ii) contacting the surface of the substrate with a composition comprising first polymerizable monomers and a radical forming initiator; (iii) initiating polymerization to produce a polymer of the monomers, wherein the polymer is covalently bound through the polymerizable moieties to the substrate; and (iv) contacting a surface of the polymer with second polymerizable monomers and, on at least one of the articles, a radical forming initiator, wherein the monomers and initiator diffuse into the polymer. In another embodiment the first article and/or second article comprise a polymer layer covalently attached to the substrate. In another embodiment the first article and/or second article comprise a second polymer layer grafted to the existing polymer layer.

In another aspect provided herein is a method of grafting a second polymer layer onto a first polymer layer comprising: (a) providing a first polymer layer having a surface; (b) contacting the surface with a radical forming initiator; (c) contacting the surface with polymerizable monomers; and (d) initiating polymerization, wherein polymerization produces a second polymer layer grafted onto the first polymer layer. In one embodiment the radical forming initiator and polymerizable monomers are co-applied to the surface. In another embodiment the method further comprises applying a chemical to the surface before initiating polymerization, wherein the chemical is immobilized in the second polymer layer. In another embodiment the radical forming initiator is allowed to diffuse into the surface before contacting with the polymerizable monomers. In another embodiment a chemical is applied with the polymerizable monomer, wherein the chemical is immobilized in the second polymer layer. In another embodiment the method further comprises removing un-polymerized material from the surface.

In another aspect provided herein is a microfluidic device comprising a first layer and a second layer, wherein the first layer and the second layer each comprise a substrate having a polymer layer attached to the substrate, wherein the polymer layers are bonded to each other; and wherein the device comprises one or more microfluidic channels passing through the grafted polymer layers and opening through ports in the grafted polymer layers. In one embodiment the layers are bonded through a polymer network that interpenetrates the polymers in the first and second layers. In another embodiment the layers are covalently bonded. In another embodiment the microchannel comprises one or more features selected from pillars, weirs, reservoirs, segment barriers, and walls. In another embodiment the device comprises a plurality of intersecting or non-intersecting microchannels. In another embodiment the device is in the form of a microshunt having a long dimension between about 1 mm and about 10 mm, and a short dimension between about 0.1 mm and about 1 mm, and wherein one or more of the microchannels traverse the length of the micro-shunt and open at ports positioned at opposite ends of the long dimension. In another embodiment the microchannels have a total resistance between 1e13 and 2.3e14 Pa*s/mA3 (e.g. a resistance between 2.5e13 and 4.5e13). In another embodiment the device comprises a plurality of microfluidic channels, wherein the channels have inlets on the same side of the device and outlets on the same side of the device. In another embodiment the polymer layer comprises a chemical entity or pharmaceutical that is elutable into a microchannel when the channel is filled with water.

In another aspect provided herein is a micro-shunt comprising a non-biofouling polymer sandwiched between solid substrates of a bio-integrative material, wherein the polymer comprises one or more microchannels having cross-sectional areas of between 100 square microns and 10,000 square microns, and having inlets and outlets opening from the polymer, wherein the micro-shunt has an elongate shape having length between about 0.5 mm and about 500 mm, and a width between about 0.5 mm and about 10 mm. In one embodiment the one or more microchannels is a plurality of microchannels. In another embodiment the micro-shunt comprises an aperture between the substrates. In another embodiment the micro-shunt is not tubular in shape.

In another aspect provided herein is a method comprising: a) inserting a device or a shunt as described herein into a body part that contains fluid so that the inlet of the microchannel is positioned in a cavity of the body part and the outlet is positioned outside of the body part; and b) draining the liquid from the body part through the microfluidic channel. In one embodiment the device or shunt is inserted into an intraocular space, intracranial space, subcutaneous space, intraabdominal space, intravesical space, peritoneal cavity, right atrium of the heart, pleural cavity, cisterna magma, subgaleal space, sinus cavity, middle ear, intra-renal space. In another embodiment the device or shunt is inserted such that an inlet is positioned inside a body and an outlet is positioned outside the body.

In another aspect provided herein is a method of treating glaucoma comprising: a) inserting a shunt into an eye such that an inlet of the shunt is positioned in the intraocular space and an outlet of the shunt is positioned outside the eye, wherein the shunt comprises a laminated structure having rigid layers of a biocompatible material and an inner polymer layer comprising a hydrogel having anti-biofouling properties and at least one channel through the polymer layer and communicating with the inlet and the outlet; and b) draining the liquid from inside the eye to outside the eye.

### BRIEF DESCRIPTION OF THE DRAWINGS

FIG. 1 shows steps in the manufacture of a microfluidic device of this disclosure.

FIG. 2 shows a method of functionalizing a solid PET substrate with a silane.

FIG. 3 shows a method of functionalizing a PET surface with a functionalized silane.

FIG. 4 shows a method of making a silicon mold coated with polyvinyl alcohol.

FIG. 5 shows a method of making a PDMS mold.

FIG. 6 shows initiation of polymerization in which a photoinitiator, 2-hydroxy-2-methylpropiophenone forms an intermediate with triethylene glycol dimethacrylate.

FIG. 7 shows a coupling reaction through which a PEG dimethacrylate polymerizes with an acryl silane attached to a PET surface.

FIG. 8 shows a cross-linked polymer covalently attached to a PET surface by a polymerization reaction as described herein.

FIG. 9 shows a method of making two substrate-backed polymeric articles, and bonding the two articles with an interpenetrating network polymer.

FIG. 10 shows a method of making two substrate-backed and partially cured polymeric articles, and bonding the two articles by curing.

FIG. 11 shows a method of grafting a polymerizable monomer to the surface of a substrate in which photointiator is interpenetrated into the substrate prior to UV photopolymerization.

FIG. 12 shows a method of grafting a polymerizable monomer to the surface of a polymer layer of a polymeric article in which photointiator and monomer are interpenetrated into the substrate and a solvent is used to draw the diffused monomer and photoinitiator to the surface.

FIG. 13 shows a method of grafting a secondary polymerizable monomer onto the surface of a partially cured polymeric article.

FIG. 14 shows a pattern of a mold to make eight microdevices on a single polymeric device. The figure also shows the resulting devices after cleavage from the master piece.

FIG. 15 shows an exploded view of an aqueous drainage device. The device comprises two PEG polymer layers, each backed by a layer of PET. One of the PEG layers include microfeatures, in this case microchannels. Bonding of the PEG layers to each other seals the channels.

FIGS. 16A and 16B show two versions of a micro-shunt of this disclosure. Both are chip shaped and have two microchannels opening at opposite ends of the chip. In addition, each device includes an aperture through the device to facilitate bio-integration.

FIG. 17 shows a flow diagram of aqueous humor dynamics with Brown glaucoma implant.

FIG. 18 shows internal ocular pressure as a function of flow rate.

### DETAILED DESCRIPTION

### I. Introduction

This disclosure provides microfeatured polymeric articles (devices) and methods of making and using them.

Certain polymeric devices of this disclosure are microfluidic devices configured to function as aqueous drainage devices. Such devices can have a sandwich configuration in which a polymer layer is sandwiched between two solid substrate layers. The polymer layer has one or more inlets and one or more outlets disposed at opposite edges of the sandwich. Inlets and outlets communicate with each other through microfluidic channels disposed in the polymer layer. The polymer layer can be bonded to the substrate layers through various methods. The polymer layer, itself, can be formed of two initial polymer layers which are bonded to each other by various methods including, for example, grafting, or curing of partially cured polymer layers. In certain embodiments the substrate layers comprise a bio-integrative material and the polymer layer has anti-biofouling properties. Such devices can be used as micro-shunts, inserted into a body part and transporting liquid from one location to another. In one embodiment, the body part is the eye, and the device drains liquid from inside the eye to outside the eye.

This disclosure also provides methods of bonding a first polymer to a second polymer. In one embodiment, methods of bonding a first polymeric article to the second polymeric article involve grafting with an interpenetrating polymer network. In general, a polymerizable monomer and the radical forming initiator are allowed to penetrate surfaces of one or both of the polymers. A solvent can then be used to draw the polymerizable monomer and the radical forming initiator to the surface or surfaces. The surfaces of the polymers are put in contact with one another polymerization is initiated. For example, the radical forming initiator can be a photoinitiator. Polymerization can be initiated by exposure to light. Polymerization of the polymerizable monomers produces a cross-linked polymer network that interpenetrates both polymeric articles, thereby bonding the polymeric articles together. When surfaces of one or both of the polymeric articles include microfeatures, such as microchannels, this method produces a tight bond that seals the microfeatures closed without gaps between the polymer layers and without deforming the shape of the microfeatures.

Another method of bonding two polymeric articles together involves creating two partially cured polymeric articles, putting surfaces of the two articles into contact and completing the curing process. This produces polymer layers that are directly bonding with each other.

Methods of this disclosure are useful for bonding, e.g. grafting, polymeric articles together for any purpose or the ultimate device. Also, devices of this disclosure can be made by methods other than bonding two polymer layers together. For example, polymeric articles that may or may not be shaped as layers and that may or may not themselves be attached to substrates can be bonded together by the methods disclosed herein. Similarly, microfeatures, such as channels, can be introduced into polymers by other methods, such as 3-D laser etching.

Method and articles of this disclosure shall be described in more detail by way of example.

### II. Methods of Making Polymeric Articles

Certain polymeric articles of this disclosure are configured as a sandwich comprising outer substrate layers and inner polymer layers. The substrate layers are attached to the polymer layers and the polymer layers, comprising micro features such as micro-channels, are attached to each other. This disclosure provides a plurality of different methods for attaching substrate to polymers and polymers to each other. These methods can be mixed in the fabrication of a single article.

Methods of attaching a substrate to polymer include, without limitation, (1) covalently attaching the polymer to a surface of the substrate through the polymerizable moieties, and (2) diffusion grafting. Methods of attaching polymer layers to each other include, without limitation, (1) grafting and (2) curing of partially cured layers.

A. Polymers

Polymers used in the methods and devices of this disclosure can be any polymeric material suitable for the use to which is to be put. In certain embodiments, the polymer is a hydrogel. Hydrogels are water insoluble network polymers with some dispersion of water throughout. Such networks are suited for penetration by monomers in which interpenetrating polymer networks can be formed. The hydrogel can be, without limitation, a poly-PEG acrylate or a polyacrylamide.

Devices of this disclosure can comprise two substrate-back polymer layers which are bonded together. The polymer on either of the two articles can be the same polymer or different polymers. The polymers can be homopolymers or a co-polymers.

“Polymerizable monomer” refers to a monomer having a polymerizable moiety. Polymers can be formed by polymerizing polymerizable monomers. “Polymerizable moiety” refers to a chemical moiety capable of undergoing a polymerization reaction. Polymerizable moieties can comprise C═C groups (“sp2-hydridized carbon atoms”). Examples of polymerizable moieties includes compounds with terminal ethylene groups, such as acrylates (e.g., methacrylates, acrylamides, etc.). Examples of polymerizable monomers include PEG acrylates. Polymerizable monomers having a plurality of polymerizable moieties can form cross-linked polymers. Accordingly, di-methacrylate monomers are useful in the polymer is described herein.

The polymerizable monomers include, without limitation, PEG-acrylates, acrylamides, acrylates, allyls, vinylics or styrenics. In one embodiment, the polymerizable monomer can comprise a polymerizable polyethylene glycol (“PEG”) such as an acrylate-PEG-acrylate. PEG monomers with, e.g., 3, 4, 5, 6, 7, 8 or 9 ethylene groups are useful, such as triethylene glycol dimethacrylate. The polymerizable monomers can comprise a zwitterion capable of free radical polymerization. The polymerizable monomers are selected from N-(2-methacryloyloxy)ethyl-N,N-dimethylammonio propanesulfonate (“SPE”), N-(3-methacryloylimino)propyl-N,N-dimethylammonio propanesulfonate (“SPP”), 2-(methacryloyloxy)ethylphosphatidylcholine (“MPC”), and 3-(2′-vinyl-pyridinio)propanesulfonate (“SPV”)).

In some embodiments, the polymerizable monomers comprise heterodisperse polyethylene glycol monomers. For example, the monomers can comprise triethylene glycol dimethacrylate and a higher molecular weight PEG (PEGMA, Mn=500) in a ratio from, for example, 5:1 to 20:1, e.g., about 9:1. By adjusting the ratio the artisan can modulate physical properties of the polymer (such as, durometer, swell ratio, flexibility, modulus, etc.). This two-part polymer solution can impart greater anti-biofouling properties. In other embodiments polymerizable monomers can further comprise a hetero bi-functional monomer such as PEG-methacrylate.

Polymerization typically will require a radical forming initiator. The radical forming initiator can be, for example, a photo initiator or a thermal initiator. Photo initiators include, for example, 2-hydroxy-2-methylpropiophenone, benzoin ethers, benzyl ketals, α-dialkoxy-alkyl-phenones, α-hydroxy-alkyl-phenones, α-amino alkyl-phenones, acyl-phosphine oxides, benzophenones, benzoamines, thioxanthones, thioamines and titantocenes. Thermal initiators include, for example, tert-Amyl peroxybenzoate, 4-4-Azobis(4-cyanovaleric acid), 1-1′-Azobis(cyclohexanecarbonitrile), 2-2′-Azobisisobutyronitrile, Benzoyl peroxide, 2,2-Bis(tert-butylperoxy)butane, 1,1-Bis(tert-butyl peroxy)cyclohexane, 2,5-Bis(tert-butylperoxy)-2,5-dimethylhexane, 2,5-Bis(tert-Butylperoxy)-2,5-dimethyl-3-hexyne, Bis(1-(tert-butylperoxy)-1-methylethyl)benzene, 1,1-Bis(tert-butylperoxy)-3,3,5-trimethylcyclohexane, tert-Butyl hydroperoxide, tert-Butyl peracetate, tert-Butyl peroxide, tert-Butyl peroxybenzoate, tert-Butylperoxy isopropyl carbonate, Cumene hydroperoxide, Cylcohexanone peroxide, Dicumyl peroxide, Lauroyl peroxide, 2,4-Pentanedione peroxide, Peracetic acid, Potassium persulfate.

Accordingly, a solution comprising polymerizable monomers and a radical forming initiator, when placed in contact with the functionalized substrate and subject to polymerization, produces a polymer covalently coupled to the substrate surface.

B. Substrates

Substrates which will form the outer layer of the sandwich, or backing, are provided. The substrates will be solid and typically rigid. For example, the solid substrate can comprise a material selected from polyethylene terephthalate (“PET”), Poly(methyl methacrylate) (“PMMA”), Thermoplastic polyurethane (“TPU”), Polyurethane (“PU”), Polycarbonate (“PC”), Polyvinyl chloride (“PVC”), Polyvinyl alcohol (“PVA”), Polystyrene (“PS”), Polyethylene (“PE”), Polysulfone, Polyethersulfone (“PES”), Sulfonated Polyethersulfone (“SPES”), Polybutylene terephthalate (“PBT”), Polyhydroxyalkanoates (“PHAs”), Silicone, Parylene, Polyproylene (“PP”), Fluoropolymers, Polyhydroxylethylmethacrylate (“pHEMA”), Polyetherketoneketone (“PEKK”), Polyether ether ketone (“PEEK”), Polyaryletherketone (“PAEK”), Poly(lactic-co-glycolic acid) (“PLGA”), Polytetrafluoroethylene (“PTFE”), or Polyvinylidene fluoride (“PVDF”) and Polyacrylic acid (“PAA”).

C. Molds

Polymerization can proceed on molds. First and second molds can be provided on which the polymers will be cast. The molds can comprise a solid material on which the polymer can be removed after casting. Such materials include for example, silicon, polydimethyl siloxane (“PDMS”), SU-8 epoxy, parylene, PTFE, dry film photoresist, or glass. The mold can be coated with a lubricant to assist removal of the polymer. For example, the lubricant can be polyvinyl alcohol (“PVA”). One or both of the molds can have one or more microfeatures on its surface that will be imparted to the polymer. “Microfeature” refers to a feature having a dimension in one aspect of no greater than 1000 microns. A microfeature can be, for example, a microchannel, or a compartment. Accordingly, a microfluidic channel refers to a fluidic channel having an aspect, such as a diameter, no greater than 1000 microns. The microchannel can comprise one or more features, e.g., pillars, weirs, reservoirs, segment barriers and walls. Microfeatures on the mold can be the inverse of features to be imparted. So, for example, a channel can be imparted to the polymer by including a ridge on the mold by adding a material to the surface, (e.g., a raised ridge) or etching a surrounding valley (troughs which define a ridge between them). In some embodiments the mold comprises a plurality of microfeature patterns, each pattern for one of a plurality of microdevices.

Other embodiments the feature is a macrofeature, that is, a feature having no dimension 1000 μm or less.

A method of producing a polymeric article can comprise filling the mold with a solution comprising the polymerizable monomers and with a radical forming initiator, overlaying the composition with the solid substrate comprising polymerizable moieties on its surface, optionally aligning the mold and the solid substrate, and initiating polymerization.

After polymerization is complete, the polymeric article, in this case comprising both a polymer layer and a solid substrate backing, can be removed from the mold. Two articles can then be bonded together using the methods described herein, e.g., through grafting with an interpenetrating polymer network.

The mold may comprise a silicon wafer with a diameter between 2 inches and 16 inches, e.g. about 4 inches. Alternatively, the mold may consist of a patterned layer of PDMS on a glass substrate, wherein the glass substrate has a diameter between 1 inch and 12 inches, and a width between 2 inches and 16 inches, e.g., 4 inches by 4 inches.

### III. Attachment of Polymers to Substrates

A. Covalent Attachment of Polymers to Substrates with Surfaces Having Polymerizable Moieties

1. Fully Cured Polymeric Articles

Certain polymeric articles of this disclosure comprise a polymer layer attached to a substrate layer through a polymerization reaction. A substrate having a surface comprising polymerizable moieties is provided for this purpose.

The surface of the solid substrate may not naturally comprise polymerizable moieties. In this case, the surface can be functionalized to incorporate such moieties. For example, one can functionalize a polymeric substrate with hydroxyl groups and couple molecules with polymerizable moieties through the hydroxyl groups. Functionalizing can comprise exposing a surface to a physical or chemical treatment for surface hydroxyl activation (e.g., exposure to O₂ plasma, air plasma, nitrogen plasma, H₂ plasma, H₂+H₂O plasma), ozone, peroxide or piranha solution.

In one embodiment, a silane bearing a terminal C═C (e.g., an acryloxyalkyl silane) is coupled to the surface through the hydroxyl groups. In this way, polymerizable acrylate groups are provided to the substrate surface. One compound useful for functionalizing a surface with polymerizable moieties is 3-acryloxypropyl trichlorosilane.

In this method, the surface of the mold is covered with a solution comprising polymerizable monomers and a radical forming initiator. The mold covered with the substrate so that the surface comprising polymerizable moieties is in contact with the polymerizable solution. Then, polymerization is initiated the polymerizable monomers coupled to each other and to the polymerizable monomers on the substrate surface. The resulting polymeric article is then removed from the mold.

2. Partially Cured Polymeric Articles

Another method for making polymeric articles for bonding together involves forming articles with partially cured polymers, placing the articles in contact and then completing polymerization so that monomers in both pieces polymerize with each other, thereby binding the two pieces together. Such a method can comprise providing a first article and a second article, wherein the first article and second article are each prepared by a method comprising: (i) providing a solid substrate comprising a surface comprising polymerizable moieties; (ii) contacting the surface of the substrate with a composition comprising polymerizable monomers and a radical forming initiator; and (iii) initiating polymerization to produce a polymer of the monomers for a time sufficient to initiate polymerization and to produce a partially cured polymer of the monomers, wherein the polymer is covalently bound through the polymerizable moieties to the substrate; wherein at least one of the articles comprises one or more microfeatures in a surface of the polymer.

The use of substrate backings, molds, polymerizable moieties and radical forming initiators can be similar to those described above in the production of fully cured polymers.

B. Attachment of Polymers to Polymeric Articles by Diffusion Bonding

In another embodiment, a second polymer layer is attached to a first polymer layer already bonded to a substrate. In general, this involves allowing a second polymerizable monomer, which may be the same or different than the first polymerizable monomer already polymerized on and bonded to the substrate, to diffuse into the polymer and polymerize, e.g., to a cured state, thereby forming a layer that coats the surface.

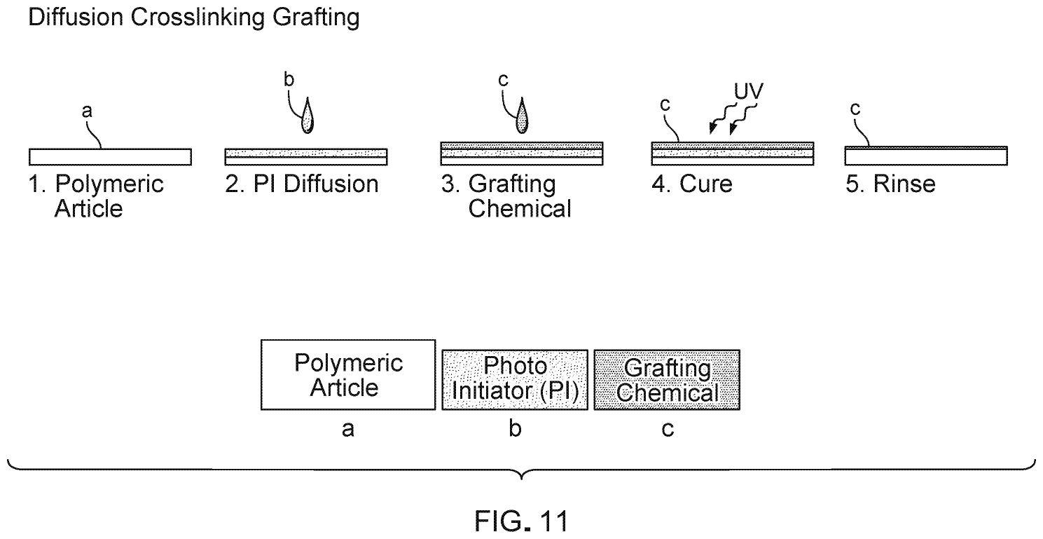

FIG. 11 shows an exemplary method of adding a second polymer layer to an existing layer of a polymeric article. A polymeric article (1) is provided. A radical forming initiator, in this case a photoinitiator, is applied to the surface and allowed to diffuse into the surface (2). A polymerizable monomer (“grafting chemical”) is applied to the surface (3). Polymerization is then initiated, in this case by exposure to UV light (4). This results in grafting of a second polymer layer to the first polymer layer. Un-polymerizable monomer is removed by rinsing (5).

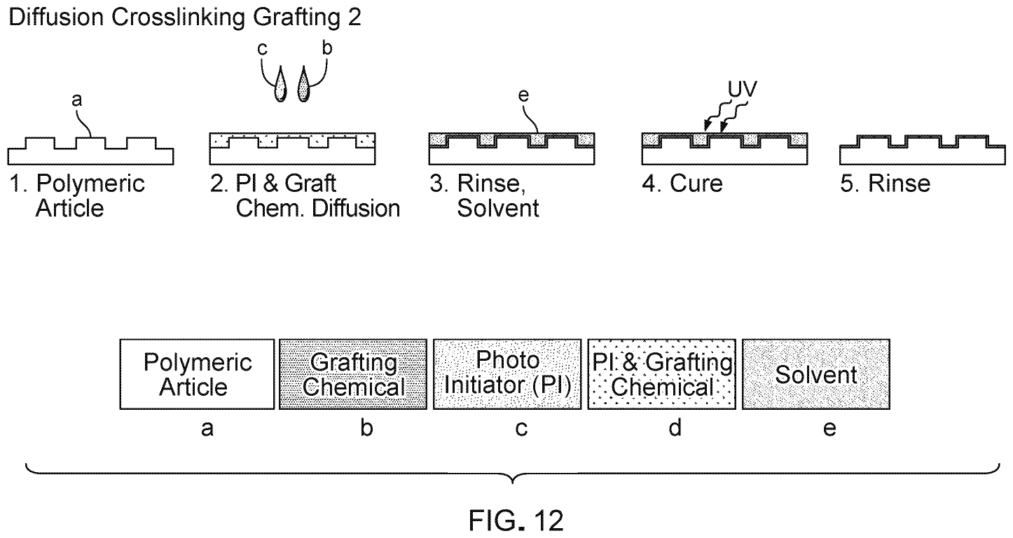

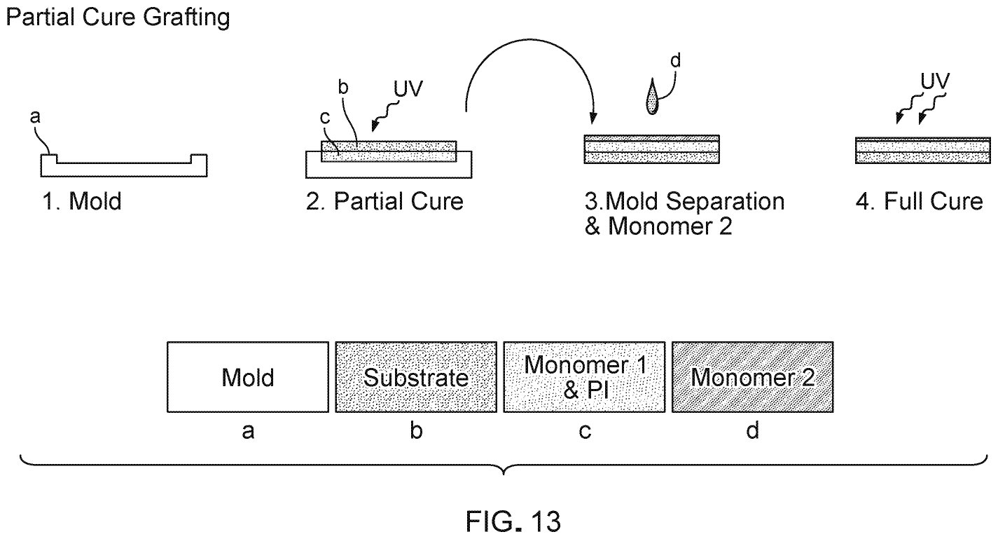

FIG. 12 shows another exemplary method of adding a second polymer layer to an existing layer of a polymeric article. In this method both and radical forming initiator and polymerizable monomers are allowed to diffuse into the surface of the existing polymer layer (2). The surface is then rinsed and a solvent is applied to the surface to draw polymerizable monomer and radical forming initiator to the surface of the article (3). Polymerization is initiated, e.g., by exposure to UV light (4). This results in the grafting of the second polymer to the surface. Un-polymerized material is washed away by rinsing (5).

### IV. Bonding Together Polymeric Articles

This disclosure also provides methods of bonding together polymeric articles. These methods can be used for the manufacture of the device is disclosed herein. The methods also can be used to bond together other polymeric articles for other purposes, for example, polymeric articles comprising polymer layers that are not attached to substrate backing.

A. Grafting Polymers with an Interpenetrating Polymer Network

A method of grafting together polymeric articles produces a polymer network that interpenetrates the polymers of the different polymeric articles, thereby bonding the articles together.

In one embodiment a method of grafting together two polymeric articles comprises: (a) providing first and second polymeric articles, each polymeric article having a polymer layer having a surface; (b) contacting the surfaces with components selected from: (i) a solvent; (ii) a radical forming initiator; and (iii) polymerizable monomers; such that each component is contacted with the surface of at least one of the polymer layers; (c) contacting the surfaces of the polymer layers with each other; (d) initiating polymerization to produce a grafting polymer of the monomers, wherein the grafting polymer interpenetrates both polymer layers, grafting the first polymeric article to the second polymeric article.

The radical forming initiator and the polymerizable monomers are provided in a solution comprising a solvent. The solution is applied to surfaces of both polymers and allowed to penetrate the polymer surfaces. The solvent can then be removed, for example, by evaporation or by heating. To bond the articles together, a solvent is applied to the surfaces of the polymeric articles to draw radical forming initiator and monomers back to the surfaces. This renders the components in each article available to react with components in the other article when the surfaces of the articles are placed in contact.

Polymerizable monomers and radical forming initiator need not be applied to surfaces of both articles. For example, the polymerizable monomers can be applied to the surface of one article and the radical forming initiator can be applied to a surface of the other article. Alternatively, the polymerizable monomers and the radical forming initiator can be applied to the surface of only one of the articles. In another alternative, and radical forming initiator can be applied to the surface of one of the articles and either polymerizable monomers or radical forming initiator can be applied to the surface all the other article.

While a solvent can be used to draw the reactive components to the surface of a polymeric article, one can also place the polymeric articles into contact before the initial solvent has been completely removed.

The choice of polymerizable monomers and radical forming initiator can be the same or different as those used in the formation of the polymeric articles. The polymer network of the articles should have a density low enough to allow penetration by the polymerizable monomers in the radical forming initiator. For example, a hydrogel can be useful in this regard.

B. Grafting of New Polymer Layers to the Inside of a Channel

In another embodiment the wall of a channel can be coated with different properties and material that of which the wall channel is comprised. This can change the chemical properties of the channel. Grafting can be performed by, for example, the diffusion crosslinking methods. For example, the method can include applying a high molecular weight PEG onto the inner lumen of a microfluidic channel to increase the anti-biofouling properties of the inner surface.

A thin film inside the channel can be added by flowing a solution containing solvent, radical initiator, and polymerizable monomer (e.g., high molecular weight PEG) through the channel, allowing diffusion into the lumen, and initiating polymerization. A monolayer can be formed by wetting the inner lumen with a monomer solution and grafting to the lumen.

C. Methods of Binding Partially Cured Polymers

Partially cured polymeric articles can be bonded together by placing surfaces of the partially cured polymers together, and re-initiating polymerization. Un-polymerized moieties on both polymeric articles react with each other. In this way, the polymers of both articles are bonded together in a single article.

D. Combination Methods

The method described above for bonding polymer layers together can be combined. For example, a first article can comprise a fully cured polymer while a second article can comprise a partially cured polymer. Polymerizable monomer and a radical forming initiator can be applied to a surface of the fully cured polymer. Surfaces of the articles can then be placed into contact and polymerization can be initiated. In this case, unreacted polymerizable moieties in the partially cured polymer react with polymerizable moieties apply to the fully cured polymer such that the partially cured polymer is bonded through a polymer network with the fully cured polymer.

### V. Cleaving Individual Devices from a Master Wafer

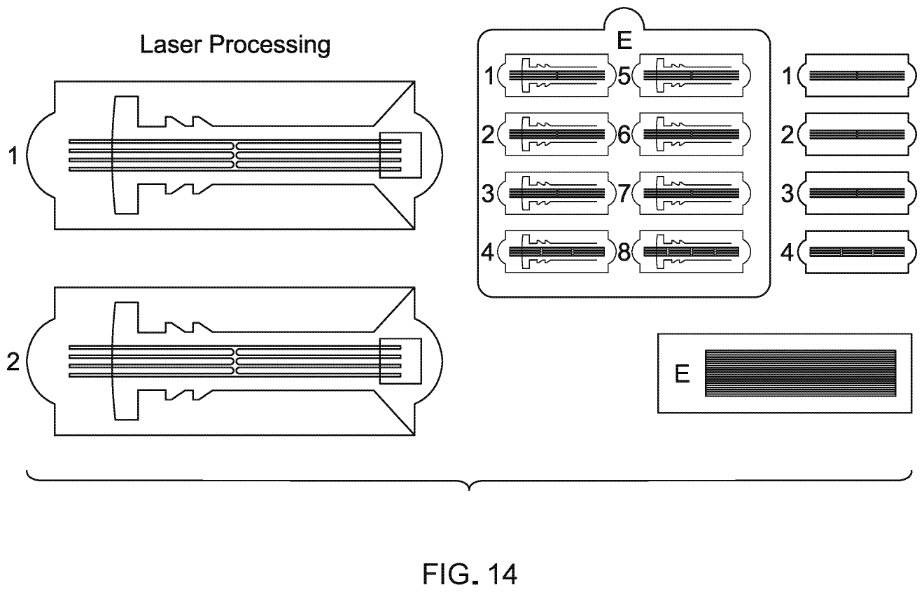

In some embodiments, the mold, and the initial article produced from it, has patterns for a plurality of microfeatured devices. Such an embodiment is depicted, for example, in FIG. 14. After casting the polymeric articles and finding them together, the wafer-like piece is cleaved into individual devices. One can cleave the wafer by mechanical means, such as sawing, or by laser cutting.

### VI. Devices

A. Microfeatured Devices

This disclosure provides polymeric devices in which a polymer layer comprises one or more microfeatures, such as microchannels. The microchannels can extend the length of the polymer layer and open onto inlets and outlets at the ends of the layer. In some embodiments the device comprises a solid backing attached to one or both sides of the polymer layer along its width. In some embodiments the polymer layer comprises two polymer layers bonded together. In such embodiments the microfeatures can be imposed on surfaces or one of one or both layers before bonding. Bonding can be achieved, for example, through an interpenetrating polymer network that interpenetrates both polymer layers. Alternatively, bonding can be achieved by providing to partially cured number two partially cured polymer layers which are placed into contact and fully cured. Both of these methods are described herein.

Devices of this disclosure can be configured for use as medical devices, that is, implantable into the body of a human or animal. In such cases, the device can comprise biocompatible materials. In certain embodiments the polymer layer comprises a hydrogel.

In certain embodiments the polymer layer contains a chemical or a pharmaceutical drug. Such compounds can be incorporated into the polymer at the polymerization stage, e.g., by diffusion bonding as described above. When the polymer is a hydrogel in which water contacted with a surface of the hydrogel, such as a microchannel, can diffuse into and out of the gel, the chemical or pharmaceutical drug can also diffuse out of the polymer and into the channel.

B. Micro Shunts

Certain articles provided in this disclosure are configured as medical devices. In particular, certain devices are configured as micro-shunts in which liquid is transported through one or more microchannels in the microfeature device. In such devices, the surface of the micro features or the entire polymer layer in which the microchannel was formed, can comprise a polymer having anti-biofouling properties. Anti-biofouling polymers resist the surface accumulation of microorganisms, plants, algae, proteins, and/or biomolecules on wetted surfaces. Several well-known polymers exhibit this property, including: PEG, Zwitterionic Polymers (contain a positive and negative charge on the same chain, e.g. Glycine, Betaine, Sulfobetaine methacrylate), poly(hydroxyfunctional acrylates), Poly(2-oxazoline)s, Poly(glycerol), Poly(vinylpyrrolidone), peptides and peptiods.

If the device is meant to function as a permanent or temporary implant, the device can comprise backing material layers of a bio-integrative material, that is, a material capable of binding to or fusing with biological materials, such as tissues. PET, Thermoplastic polyurethane (“TPU”), and polyurethane are examples of such materials. The bio-integrative property of a material can be enhanced with a localized plasma etching process in which plasma is used to roughen the surface on the nano-scale and create a more favorable surface chemistry for cells. An array of plasma gases can be used to this end. This effect can be localized by physically masking the plasma using a PDMS or silicone membrane with a specifically placed array of holes. Such devices bind to the tissue at the anchoring site and prevent cell attachment near the ends which could block the inlet/outlet.

Accordingly, provided herein is a microfluidic device comprising a first layer and a second layer covalently bonded together and one or more microfluidic channels internal to the device, wherein the first layer and the second layer each comprise a substrate and a polymer bonded to the substrate, and wherein the two layers are bonded, e.g., through a polymer network that interpenetrates the polymers in the first and second layers. The microchannel can comprise one or more features, e.g., selected from pillars, weirs, and reservoirs. In some embodiments the device comprises a plurality of intersecting microchannels. In other embodiments a plurality of the channels do not intersect.

More particularly, the device can be configured as a microshunt, wherein the microchannel opens at an inlet and an outlet in the polymer, wherein at least one of the inlet and the outlet are disposed at the one or more ends, the microchannel comprises a channel inlet and channel outlet disposed in the polymer; and the microshunt has a length between about 1 mm and about 10 mm, and a width between about 0.1 mm and about 1 mm. in some embodiments the device comprises a plurality of microfluidic channels, wherein the channels have inlets on the same side of the device and outlets on the same side of the device.

In some embodiments a micro-shunt comprises a non-biofouling polymer sandwiched between solid substrates of bio-integrative material, wherein the polymer comprises one or more microchannels having cross-sectional areas of between 100 square microns and 10,000 square microns, e.g., 1300-1600 square microns, and having inlets and outlets opening from the polymer, wherein the micro-shunt has an elongate shape having length between about 0.5 mm and about 500 mm (e.g., between about 2 mm and about 5 mm), and a width between about 0.5 mm and about 10 mm (e.g., a length between about 3-5 mm and a width between about 1-2 mm. The micro-shunt can comprise a plurality of microchannels.

Devices of this disclosure can have a chip configuration. That is, they may have a generally flat configuration in which the thickness is substantially less than the length and/or width. For example, the length to thickness ratio can be at least 5:1, at least 10:1, or at least 20:1. The width of the device is typically equal to or greater than the thickness. Typically, the top and bottom of the device will each be substantially planar and substantially parallel to one another. For example, the chip can have a substantially cuboid shape.

In certain embodiments the chip comprising an aperture through the width of the chip, that is, traversing the chip through the substrate layers and a polymer layer. Accordingly, a micro-shunt of this disclosure may have a shape which is not tubular.

### VII. Methods of Use

Micro-shunts of this disclosure are useful as medical devices to transport liquids fluids from areas of higher pressure to areas of lower pressure. Accordingly, this disclosure provides methods comprising: a) inserting a shunt of this disclosure into a body part that contains liquid so that the inlet of the microchannel is positioned in a cavity of the body part and the outlet is positioned outside of the body part; and b) draining the liquid from the body part through the microfluidic channel. For example, the shunt can be inserted into an intraocular space, intracranial space, subcutaneous space, intraabdominal space, intravesical space, peritoneal cavity, right atrium of the heart, pleural cavity, cisterna magma, subgaleal space, sinus cavity, middle ear or intra-renal space. The shunt can be inserted such that an inlet is positioned inside a body and an outlet is positioned outside the body. Alternatively, inlet and the outlet of the shunt both can be positioned inside the body.

Accordingly, this disclosure provides methods for treating glaucoma. The methods comprise inserting a micro-shunt of this disclosure into the intraocular space and draining ocular fluid through one or more microchannels in the micro-shunt from the intraocular space to outside the body. The micro-shunt can be inserted at the edge of the iris. A keratome blade can be used to make an incision in the eye. The micro-shunt will is gently pushed through the opening. When the micro-shunt comprises a backing of a bio-integrative material, the shunt can be left in place for long periods of time. Because glaucoma is characterized by increased pressure inside the eye and because the environment outside the eye is at air pressure, liquid will flow in a direction from greater pressure less pressure, that is, from the inside of the eye to the outside the eye.

Micro-channels of micro-shunts can have a resistance to flow of liquids such that pressure inside the body part is sufficient to provide flow of liquid from the shunt inlet to the shunt outlet. For example, an aqueous drainage device, a plurality of microchannels can have a hydraulic resistance between 1e13 and 2.3e14 Pa*s/m\hat{ }3 (e.g. 3.75e13 Pa*s/m\hat{ }3). Dimensions to achieve such resistance can be achieved based on algorithms provided herein.

### EXAMPLES

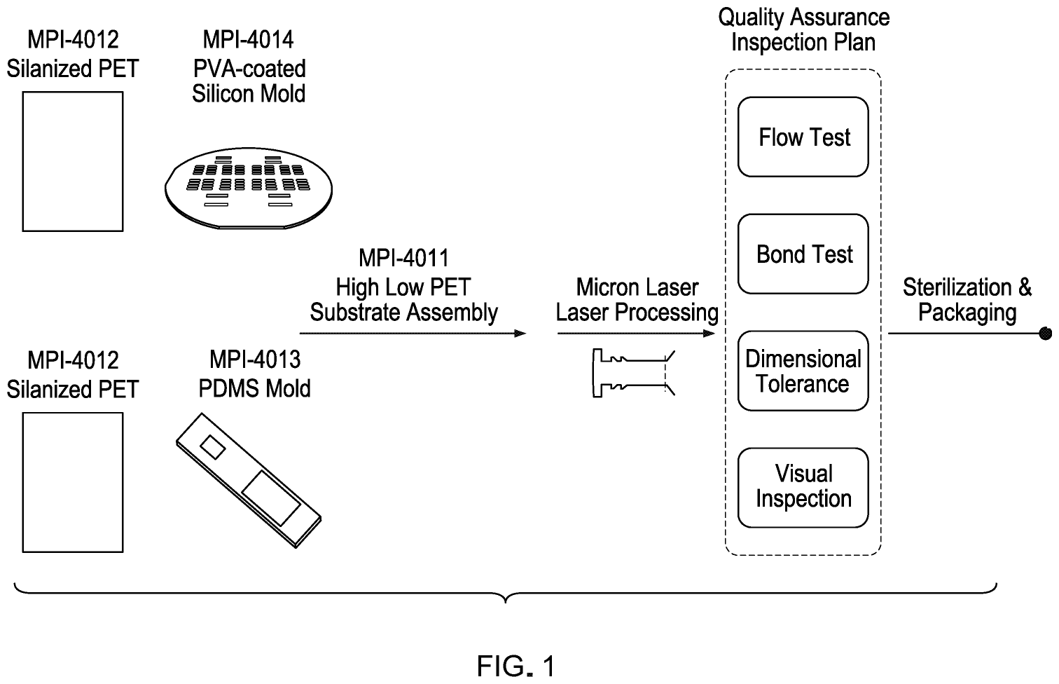

A general method of making a polymeric device of this disclosure is presented in FIG. 1. Two silanized by PET substrates are provided. Each of these is paired with a mold. In this example, one of the molds is a patterned silicon mold coated with PVA. The other mold is a non-patterned mold made of PDMS. Polymers are cast on the mold and bound to the PET substrates. After casting, the device is cut into separate pieces by laser processing. The devices proceed through a quality assurance inspection plan. Then they are sterilized and packaged.

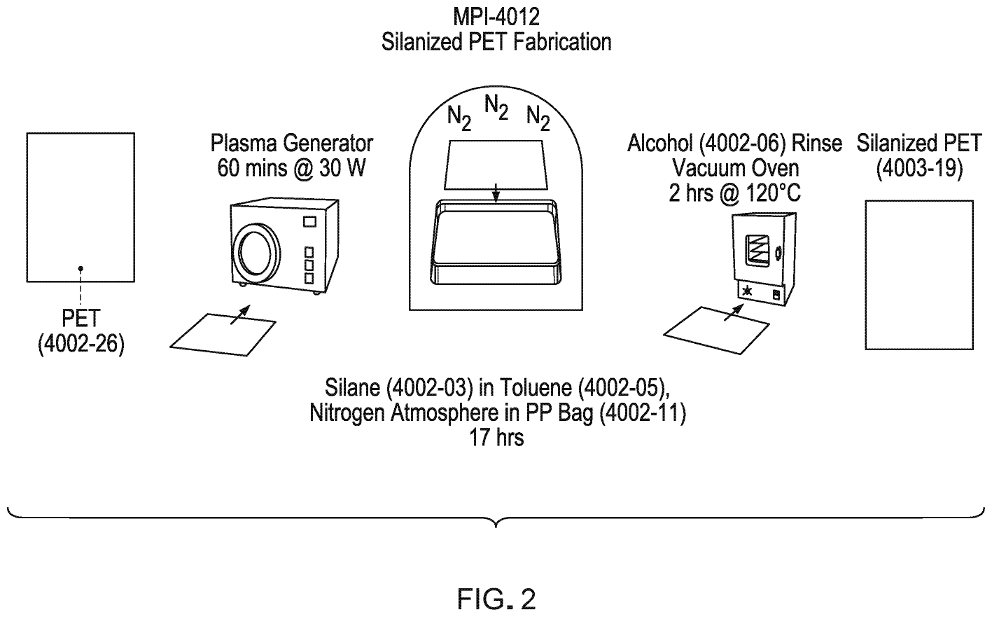

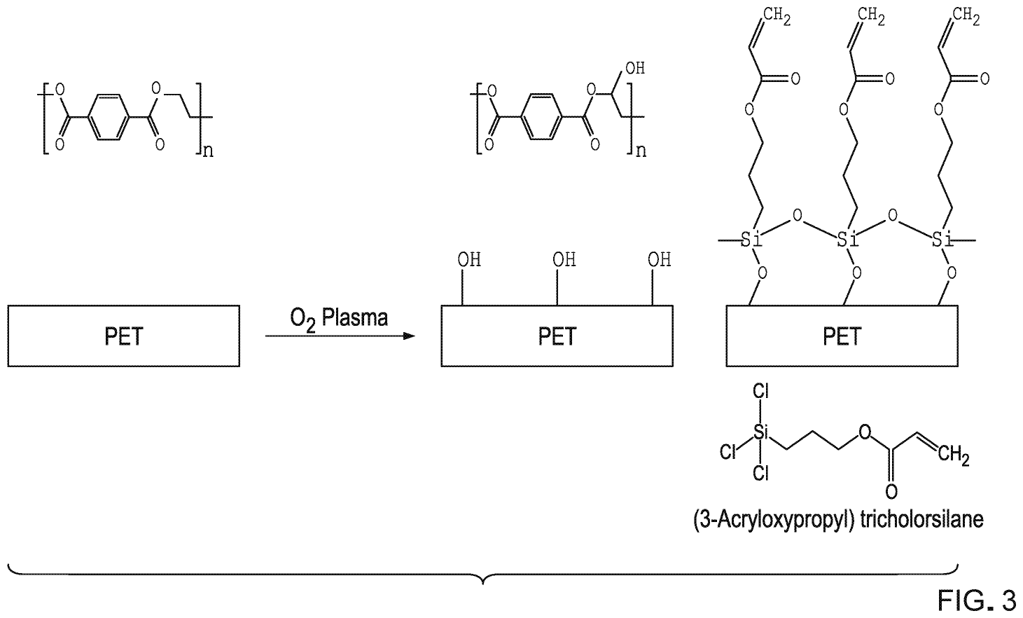

FIG. 2 shows an overview of the fabrication of a silanized substrate. A PET substrate is provided. Hydroxyl groups are generated on the PET substrate by exposure to plasma for 60 minutes at 30 W. PET with a reactive surface is contacted with a silane in toluene in a nitrogen atmosphere in a poly propylene bag for 17 hours. The functionalized substrate is rinsed with alcohol and drive in a vacuum oven for two hours at 120° the silanized PET substrate is now ready for use. FIG. 3 shows the chemistry of coupling of (3-acryloxypropyl) trichlorosilane to a reactive PET surface.

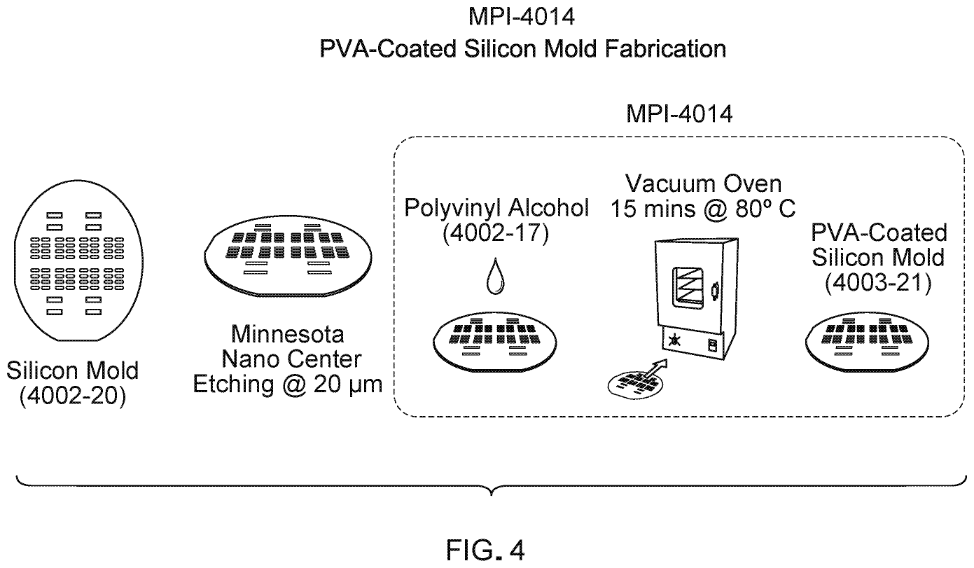

FIG. 4 shows the fabrication of a PVA coated silicon mold. A silicon mold having a plurality of micro feature patterns is provided micro features are provided on the surface by etching. A solution of polyvinyl alcohol is provided to the pattern surface of the substrate. The substrate is dried in a vacuum oven for 15 minutes at 80° C. The mold is now ready for use.

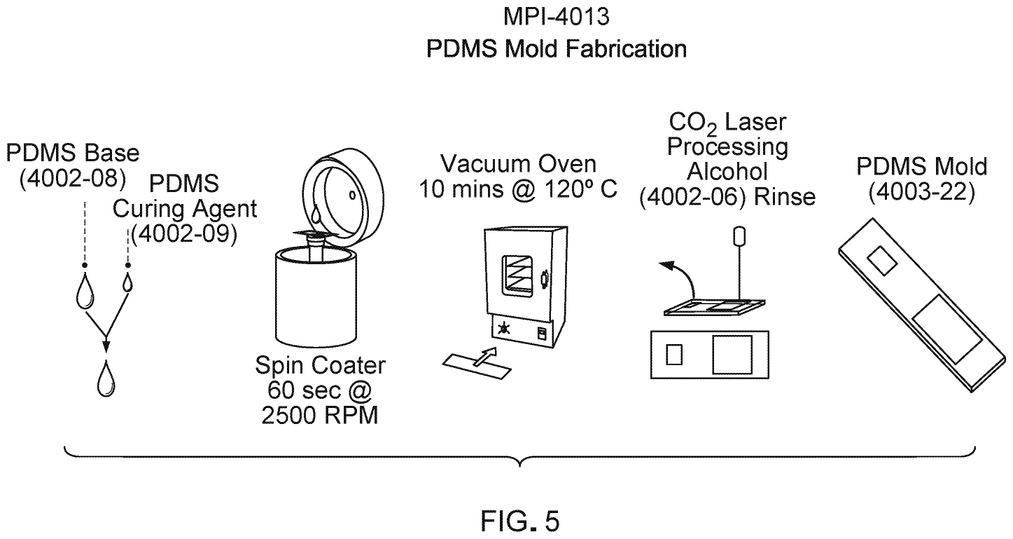

FIG. 5 shows the fabrication of a PDMS mold. A PDMS base and a curing agent are mixed to provide a solution. The solution is spread on a surface with a spin coater by spinning for 60 seconds at 2500 RPM. The resulting polymer is dried in a vacuum oven for 10 minutes at 120° C. The mold is shaped by CO₂ laser processing and an alcohol rinse. The PDMS mold is now ready for use.

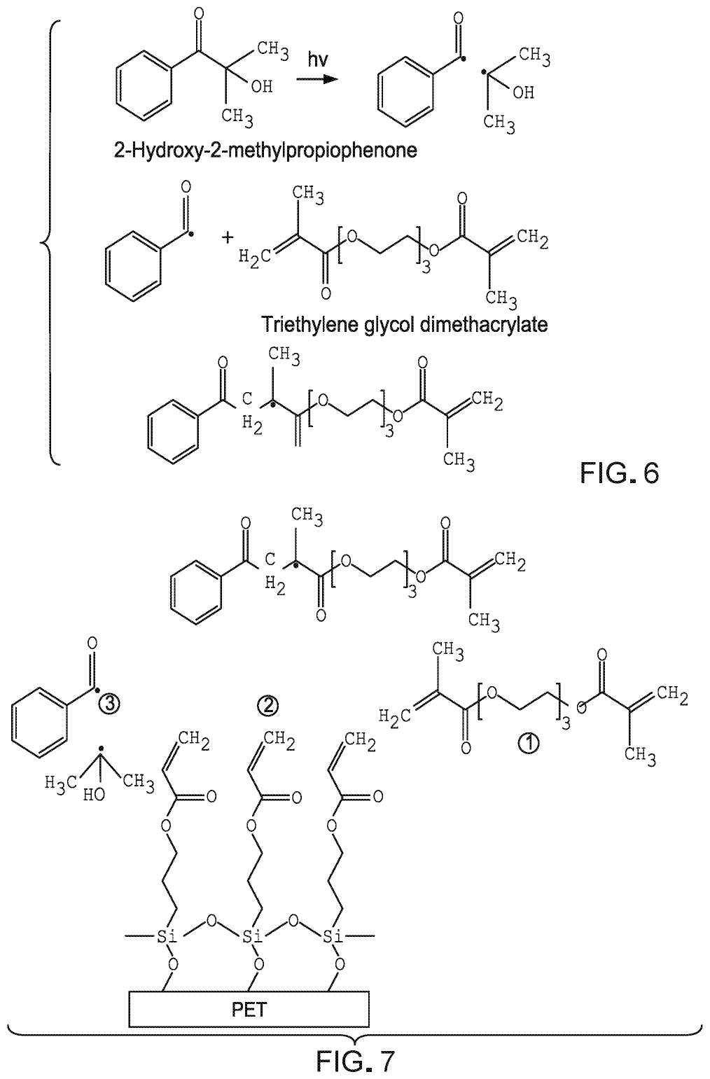

FIG. 6 shows initiation of photopolymerization. 2-hydroxy-2-methylpropiophenone, when exposed to light, produces a phenone radical. The phenone radical couples with an acrylate group of triethylene glycol dimethacrylate.

FIG. 7 shows the polymerization of triethylene glycol dimethacrylate to surface bound acryloyl siloxane.

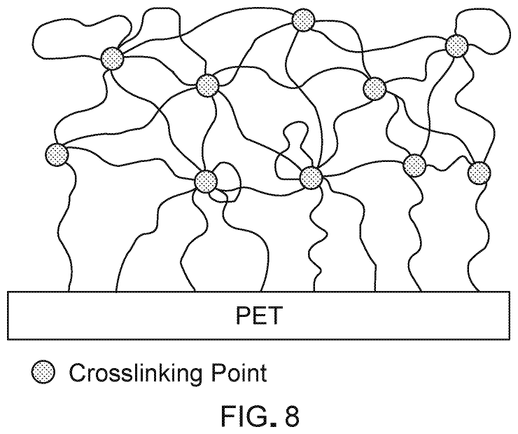

FIG. 8 shows a resulting cross-linked polymer of PEG bound to the PET substrate.

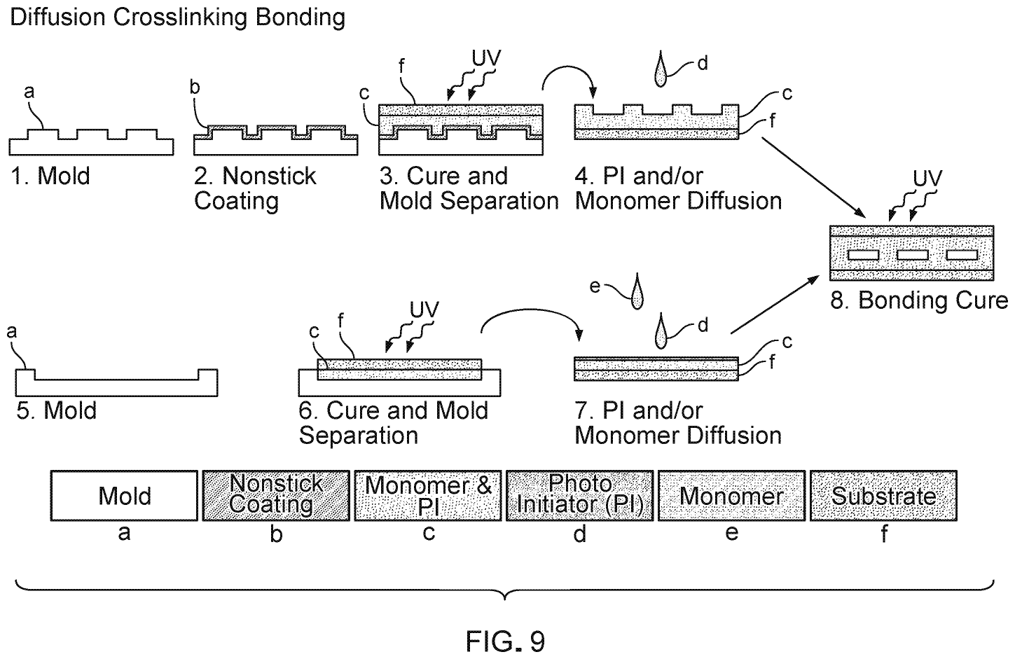

FIG. 9 shows the fabrication of the microdevice of this disclosure. A patterned mold is provided and covered with a nonstick coating, such as PVA. A polymeric article is produced by covering the mold with a solution of polymerizable monomers and a substrate having polymerizable moieties, and initiating polymerization. The polymeric article is removed from the mold and photo initiator and the polymerizable monomer are applied to the surface of the polymer and allowed to diffuse in. A similar process is used to produce a non-patterned polymeric article. The polymer layers of both articles are placed into contact and polymerization is initiated, in this case by exposure to UV light. Polymerization of the polymerizable monomers produces a polymer network that spans both polymeric articles, binding them together.

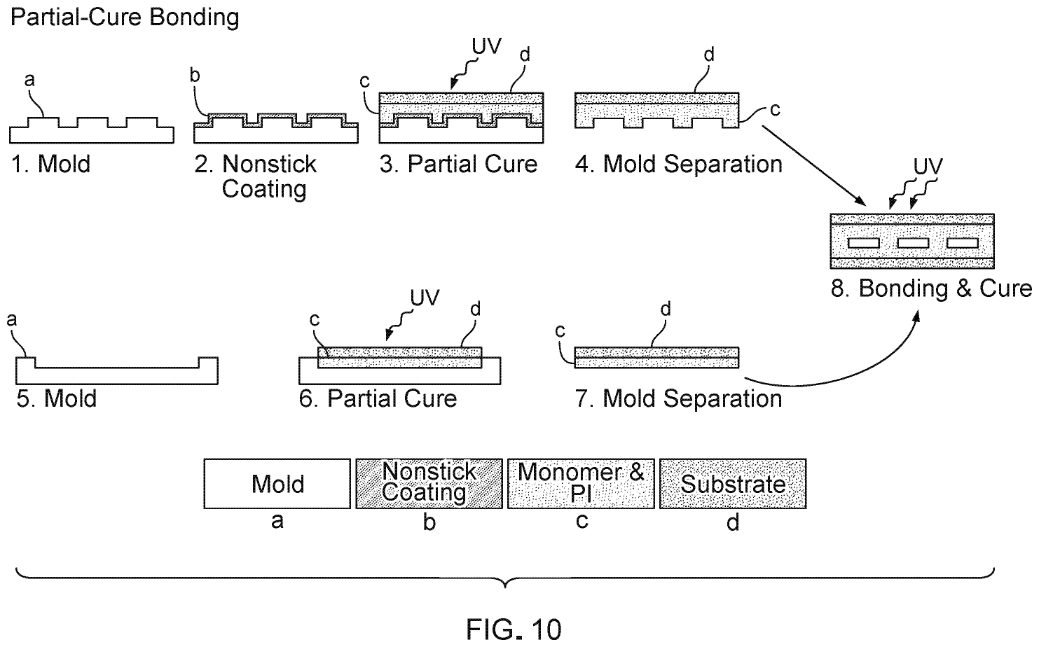

FIG. 10 shows a method of fabricating a microfluidic device by partial-cure bonding. The molds are provided. The molds are covered with polymerizable monomer and he substrate. Polymerization is initiated. However, the polymerization is stopped before curing is complete, resulting in two partially cured polymeric articles. The partially cured polymers of these two articles are put into contact and polymerization is recommenced. This results in polymerization of polymerizable monomers in both pieces with each other. This bonds the two pieces together.

FIG. 14 shows a template for making eight microfluidic devices. After fabrication on a single piece, the individual die are cut from the master, for example, by laser cutting or by sawing.

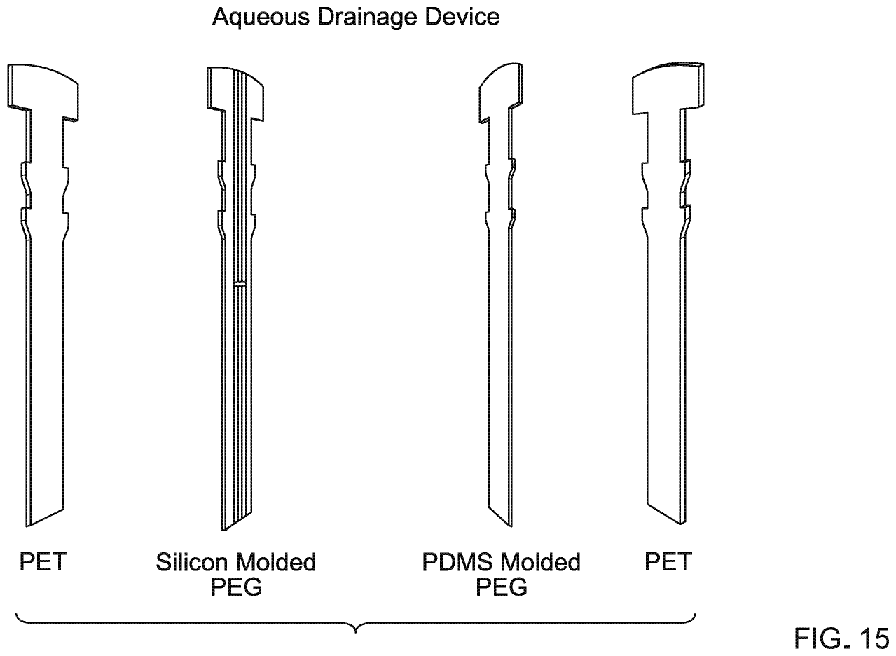

FIG. 15 shows an exploded view of an exemplary aqueous drainage device. The device comprises outer substrate layers of PET. The inner layers comprise PEG polymers. The layers are bonded together by methods described herein.

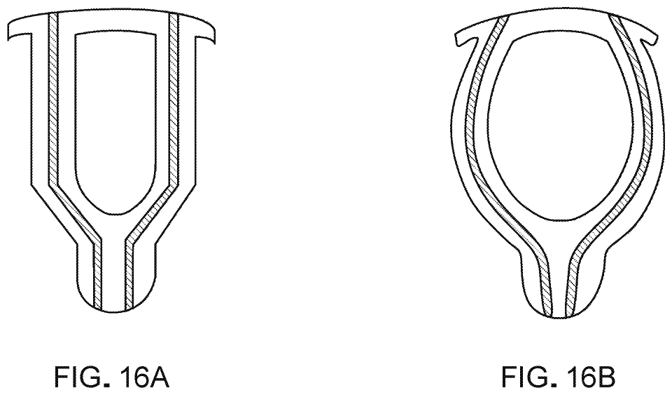

FIGS. 16A and 16B show exemplary drainage devices of this disclosure. The devices include apertures, indicated as open spaces. The open spaces provide a ring-like configuration to the device. The open space promotes bio-integration. The drainage devices allow transverse compression, allowing the devices to pass through or into an opening that is narrower than its uncompressed width. Accordingly, a friction fit can be thereby achieved.

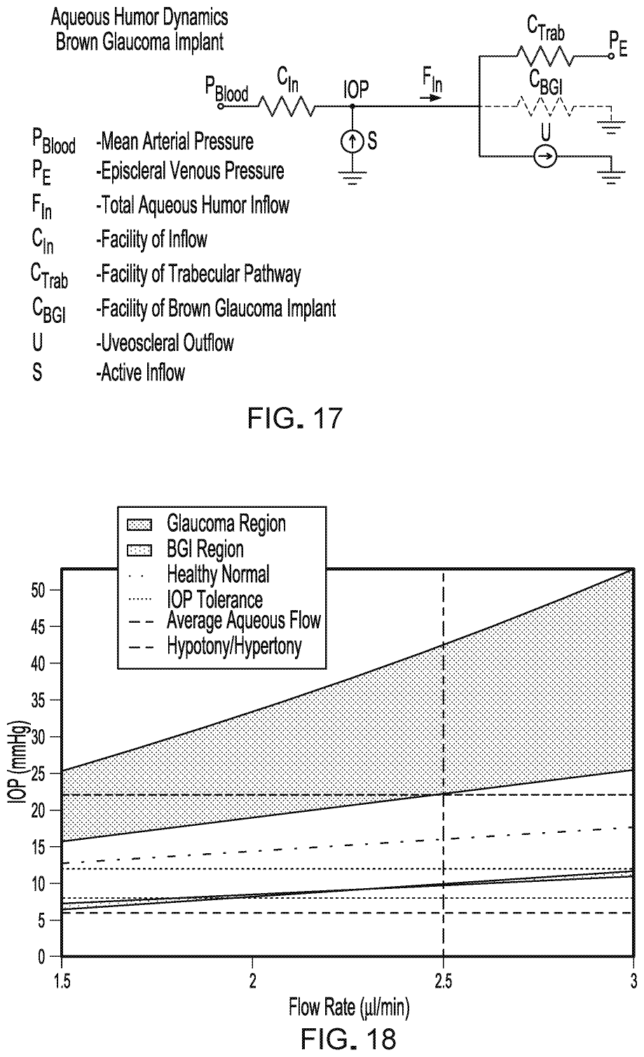

Design of intraocular shunts can implement an aqueous humor dynamics model from Alder's Physiology of the Eye 9th Edition recommended by the FDA. FIG. 17 shows a flow diagram of this model. In the determination of channel resistance, the parameters affecting outflow must be defined. Parameters described in Alder's 10th Edition that can be used include: average human episcleral venous pressure (PE)=9 mmHg, average aqueous inflow=2.5 μl/min, and uveoscleral outflow (U) in glaucoma=0.3 μl/min. Trabecular facility is calculated using the Brubaker-Modification, which accounts for the increase in trabecular resistance at high IOP. An individual's trabecular facility—from healthy to severe glaucoma—can be summarized by the quantity R0.

FIG. 18 shows a dark region of intraocular pressure values for glaucoma patients with varying levels of trabecular facility. The R0 range is from 5.35—where IOP is greater than 22 mmHg at average inflow—to 15—Alder's recommendation of maximum R0. Each figure then displays a green region of IOP values for those same patients when the Model 8C BGI is added. For reference, a blue line describing a healthy individual is added (R0=3). The resistance is set where pressure is 10 mmHg at 2.5 μl/min inflow. With these conditions, hypotony is not expected even if flow rates drop to 1.5 μl/min.

### Abbreviations

Fin—Total inflow

Fout—Total outflow

FS or S—Inflow from active secretion

FF—Inflow from filtration

FTrab—Trabecular outflow

FU or U—Ulveoscleral outflow

FBGI—BGI outflow

PBlood—Mean arterial pressure

PE—Episcleral venous pressure

IOP—Intraocular pressure

Cin—Inflow facility

CBGI—BGI facility

CTrab—Trabecular facility

Ro—Brubaker resistance

Q—Brubaker obstruction factor

IOP Dynamic Equilibrium Equations

The steady state assumption is inflow equals outflow: Fin=Fout

Components of inflow and outflow substituted: (FF+FS)−(FTrab+FU+FBGI)=0

Detailed Components:
Inflow from filtration: FF=(PBlood-IOP)
Inflow from secretion: FS=S
Ulveoscleral outflow: FU=U=0.3 μl/min (average for patients with glaucoma from Alder's
Physiology of the Eye, 10th Edition)
Trabecular outflow with Brubaker modification: FTrab=(IOP-PE)Ro+RoQ(IOP-PE)
(Q=0.008, PE=9 mmHg from Alder's 9th and 10th Edition). When IOP is low, outflow is close to non-modified flow: FTrab≈(IOP−PE)*CTrab
BGI outflow: FBGI=IOP*CBGI

Full Brubaker-Modified Equation: (PBlood-IOP)*Cin+S−(IOP-PE)Ro+RoQ(IOP-PE)-U−IOP*CBGI=0

Simplified Equation for Channel Determination: Fin=(IOP−PE)Ro+RoQ(IOP−PE)+U+IOP*CBGI

Assumptions:
Steady state—inflow equals outflow
Average inflow is 2.5 μl/min (from Alder's 10th Edition)
U=0.3 μl/min (from Alder's 10th Edition)
PE=9 mmHg (from Alder's 10th Edition)
Ro between 5.35-15 (resistances that cause IOP above 22 mmHg at average inflow);
Q=0.008 (from Alder's 9th Edition)

All publications and patent applications mentioned in this specification are herein incorporated by reference to the same extent as if each individual publication or patent application was specifically and individually indicated to be incorporated by reference.

While certain embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of making a polymeric device, the method comprising:
(a) providing:
a first article having a first surface; and
a second article having a second surface;
(b) contacting the first surface with a radical forming initiator;
(c) contacting the first surface with polymerizable monomers to create a polymer layer;
(d) attaching the second surface of the second article to the polymer layer; and
(e) initiating polymerization of the polymer layer such that the first article is bonded to the second article.

2. The method of claim 1, wherein the polymer layer comprises a hydrogel.

3. The method of claim 1, wherein the polymer layer has anti-biofouling properties.

4. The method of claim 1, wherein the polymer layer comprises poly-PEG acrylate, polyacrylamide, poly(glycerol), poly(2-oxazoline), poly(hydroxyfunctional acrylate), poly(vinylpyrrolidone), peptides, or peptoids.

5. The method of claim 1, wherein the polymer layer comprises one or more microfeatures.

6. The method of claim 5, wherein at least one microfeature is a microchannel.

7. The method of claim 1, wherein the polymerizable monomers comprise a polymerizable polyethylene glycol (“PEG”).

8. The method of claim 7, wherein the polymerizable polyethylene glycol comprises triethylene glycol dimethacrylate.

9. The method of claim 1, wherein the first article and second article are provided by a method comprising providing a solid substrate comprising a surface comprising polymerizable moieties.

10. The method of claim 9, wherein the solid substrate comprises a material selected from polyethylene terephthalate (“PET”), Poly(methyl methacrylate) (“PMMA”), Polyurethane (“PU”), Polycarbonate (“PC”), Polyvinyl chloride (“PVC”), Polyvinyl alcohol (“PVA”), Polystyrene (“PS”), Thermoplastic polyurethane (“TPU”), Polyethylene (“PE”), Polysulfone, Polyethersulfone (“PES”), Sulfonated Polyethersulfone (“SPES”), Polybutylene terephthalate (“PBT”), Polyhydroxyalkanoates (“PHAs”), Silicone, Parylene, Polyproylene (“PP”), Fluoropolymers, Polyhydroxylethylmethacrylate (“pHEMA”), Polyetherketoneketone (“PEKK”), Polyether ether ketone (“PEEK”), Polyaryletherketone (“PAEK”), Poly(lactic-co-glycolic acid) (“PLGA”), Polytetrafluoroethylene (“PTFE”), Polyvinylidene fluoride (“PVDF”), and Polyacrylic acid (“PAA”).

11. The method of claim 9, wherein providing the solid substrate comprises functionalizing a polymeric substrate with hydroxyl groups and coupling (3-acryloxypropyl) trichlorosilane through the hydroxyl groups.

12. The method of claim 9, further comprising functionalizing the surface of the substrate by exposing the surface to a physical or chemical treatment for surface hydroxyl activation.

13. The method of claim 9, wherein at least one of the articles comprises one or more microfeatures in the polymer.

14. The method of claim 1, wherein the first article and/or second article comprise a polymerizable moiety covalently attached to the substrate.

15. The method of claim 1, wherein providing at least one article comprises providing a mold optionally having one or more microfeatures, overlaying the mold within the polymer layer, optionally aligning the mold and the solid substrate and initiating polymerization, and removing the mold.

16. The method of claim 15, wherein the mold comprises polyvinyl alcohol (“PVA”) coated silicon, polydimethyl siloxane (“PDMS”), SU-8 epoxy, parylene, PTFE, dry film photoresist, or glass.
